(19) United States
(12) Patent Application Publication
(10) Pub. No.: US 2023/0371452 A1
(43) Pub. Date: Nov. 23, 2023

(54) COMPUTER-IMPLEMENTED METHOD FOR DETERMINING PLANT DATA AND/OR FOR ISSUING TREATMENT INSTRUCTIONS IN HYBRID BREEDING

(71) Applicant: BASF SE, Ludwigshafen am Rhein (DE)

(72) Inventor: Uwe BUCKENAUER, Mannheim (DE)

(21) Appl. No.: 18/031,370

(22) PCT Filed: Oct. 15, 2021
(86) PCT No.: PCT/EP2021/078660

§ 371 Date: Apr. 12, 2023

(30) Foreign Application Priority Data
Oct. 15, 2020 (EP) 20202026.9

(51) Int. Cl. A01H 1/04

(52) U.S. Cl. CPC A01H 1/04

(57) ABSTRACT

Computer-implemented method for determining plant data and/or for issuing treatment instructions in hybrid breeding, comprising the following steps: providing a first computer-implemented growth model for a male parent plant, wherein the first computer-implemented growth model is configured to output time-resolved growth data of the male parent plant dependent on at least a first parameter (S10); providing a second computer-implemented growth model for a female parent plant, wherein the second computer-implemented growth model is configured to output time-resolved growth data of the female parent plant dependent on the at least first parameter (S10); inputting or providing at least one parameter into the first growth model to determine pollination phase data of the male parent plant (S20); inputting or providing the at least one parameter into the second growth model to determine flowering phase data of the female parent plant (S20); providing pollination phase data of the male parent plant and flowering phase data of the female parent plant based on the at least first parameter (S30).

### TECHNICAL FIELD

The present disclosure relates to a computer-implemented method for determining plant data and/or for issuing treatment instructions in hybrid breeding, to an use of a computer-implemented growth model for a parent plant, to an use of geographical location data of a field at which a hybrid breeding is planned in such a method, to a system for determining plant data and/or for issuing treatment instructions in hybrid breeding and a computer program element.

### TECHNICAL BACKGROUND

Hybrid plants are well known in the state of the art in the agricultural area. Hybrid plants show advantageous properties over non-hybrid plants, e.g. higher vigor or higher resilience against adverse environmental conditions. Hybrid plants are generated from two parent lines of a particular plant.

### SUMMARY OF THE INVENTION

It is an object of the present invention to provide a method supporting hybrid breeding. These and other objects, which become apparent upon reading the following description, are solved by the subject matter of the independent claims. The dependent claims refer to preferred embodiments of the invention.

According to a first aspect of the present disclosure, a computer-implemented method for determining plant data and/or for issuing treatment instructions in hybrid breeding is provided, comprising the following steps: providing a first computer-implemented growth model for a male parent plant, wherein the first computer-implemented growth model is configured to output time-resolved growth data of the male parent plant dependent on at least a first parameter; providing a second computer-implemented growth model for a female parent plant, wherein the second computer-implemented growth model is configured to output time-resolved growth data of the female parent plant dependent on the at least first parameter; inputting or providing the at least one parameter into the first growth model to determine pollination phase data of the male parent plant; inputting or providing the at least one parameter into the second growth model to determine flowering phase data of the female parent plant; providing pollination phase data of the male parent plant and flowering phase data of the female parent plant based on the at least first parameter.

The term plant data is understood broadly in the present case and comprises e.g. growth data, pollination time and duration, flowering time and duration of special plants. The term treatment instructions is also understood broadly in the present case and comprises all aspects related to treatment processes like e.g. sowing time, sowing density, application rate of growth regulators and herbicides, artificial flower fertilization steps (i.e. for example artificial wind flux by using drones), application of soil fertilizer, application of fungicides, application of insecticides and/or harvesting times. The term hybrid breeding is defined in the present case by using two parent lines of a particular plant species, preferably a male line and a female line, for generating of a hybrid plant.

A male parent plant according to the present disclosure may be defined as a plant that provides male genetic material. A female parent plant according to the present disclosure may be defined as a plant that provides female genetic material.

In one example, a male parent plant may be a monoecious plant with both male reproduction organs and female reproduction organs, and a female parent plant may be a monoecious plant also with both male reproduction organs and female reproduction organs. In this example the male reproduction organs of the female parent plant may be sterilized by use of a heterologous cytoplasm or a mutated gene. Thus, only the female reproduction organs of the female parent plant may express fertility. In this example the first computer-implemented growth model may relate to the male parent plant, which in this example comprises both male and female reproduction organs, which both provide fertility. In this example the second computer-implemented growth model relates to the female parent plant, where the male reproduction organs have been sterilized and only the female reproduction organs provide fertility. Therefore, in this example self-pollination may occur at the male parent plants and therefor may lead to none hybrid seeds at the male parent plant. However, in this example self-pollination may not occur at the female plants, since the female plants, with sterilized male reproduction organs, can only be pollinated from the male parent plants and therefor lead to hybrid seeds. In this example, hybrid seed is the seed harvested on the female parent plants.

In a further example also the male parent plant may be either modified to dominantly express male fertility, i.e. in case of a monoecious plant, or only has male reproduction organs, i.e. in case of a dioecious plant. In this example the female parent plant may be a plant, which is either modified to dominantly express female fertility, i.e. in case of a monecious plant, or which has only female reproduction organs, i.e. in case of a dioecious plant. The modification of a plant to express dominantly male or female fertility may be achieved by the use of heterologous cytoplasm or a mutated gene or any other applicable/appropriate method. Hence, cross pollination is between a male parent plant and female parent is easy to achieve, as no self-pollination can occur.

Therefore, the present disclosure is not limited to dioecious plants or monoecious plants, but can be performed with both plant varieties. Hence, the term male parent plant and the term female parent plant comprises both dioecious plants and monoecious plants. In case of a monoecious plant such as wheat, rice or oilseed rape, the term female parent plant relates to the female parent line of the monoecious plant and the term male parent plant relates to the male parent line of the monoecious plant.

The term computer-implemented growth model describes in the present case an equation-based model (e.g. statistical model, neuronal network, physical model), which describes the growth behavior of the plants, in particular the growth model comprises time-resolved growth data for the flowering phase and for the pollination phase. The term flowering phase is defined in the present case as phase wherein the female plants flower. The term pollination phase is defined in the present case as phase wherein the male plant pollinate. The term parameter comprises in the present case values, factors, conditions of plants and/or planting related aspects like for example soil data (e.g. pH, composition like nitrogen and phosphorus content), intensity and duration of sunlight of the location, weather data (precipitation, temperature, wind), sowing time, harvesting time, diseases (e.g. fungal diseases, herbs insects, nematodes) and/or geographical location. Notably, the terms “inputting or providing” are to be understood broadly in the context of the present disclosure and includes not only a manual input by an operator but also, in particular, automated data transmission/transfer and the like. The present disclosure is based on the finding that hybrid plants have advantageous properties against conventional plants due to their high genetic diversity, but that the generating of the hybrid plant seeds requires two parent lines of a particular plant species (i.e. a male and a female parent line or male parent plant and female parent plant). This is due to Mendel's rule, that the so called heterosis effect is mostly limited to the F1-generation. Hence, hybrid plants respectively their seeds lose their heterozygote genotype successively. Consequently, the hybrid plant seeds have to be generated each time by two parent lines of a particular plant, which normally have different pollination phases and flowering phases. The different pollination phase and flowering phase result in a decreased yield of the hybrid plant seeds. Hence, it is advantageous to determine the pollination phase and flowering phase of the parent lines of the particular plant and to adapt respectively synchronize the pollination phase and the flowering phase in order to increase the yield of the hybrid plant seeds. The increased yield of the hybrid plant seeds leads to reduced production costs (due to less initial parent plants, less machine usage, smaller agricultural area, less fertilizers and pesticides). It should be noted that for every hybrid plant species different computer-implemented growth models for the female parent plant and the male parent plant exist. A further advantage of the synchronization of the pollination phase and the flowering phase is the prevention of plant diseases. For example, the infection of the plant with fungus Claviceps purpurea occurs if the synchronization of pollination phase and flowering phase is not fully achieved.

In an embodiment, the method further comprises the steps of providing plant data for the male parent plant and providing plant data for the female parent plant such that the flowering phase of the female plant corresponds to the pollination phase of the male plant. The plant data may comprise general information of the male and female parent plants (general sowing time, general flowering time, general pollination time, content of the plants, light requirement, temperature requirement, humidity requirement, nutrition requirement, plant species etc.). The plant data may be advantageous for a user to get a first information of the plants (e.g. approximate information of sowing time of the female parent plant and the male parent plant, in order to synchronize flowering and pollination phase, suitability combination plant/field). The plant data may serve as an overview in a planning phase for a user (e.g. farmer, breeder).

In an embodiment, the first parameter are geographic location data at which hybrid breeding is planned. The geographic location data may be acquired by a user input of the farmer or by a GPS signal from a GPS or also maybe by a mobile phone. The geographic data may be associated with weather data, climate data, soil data stored in a data base, etc. Hence, it is advantageous possible to efficiently determine with little user input the pollination phase and flowering phase of the male/female parent plants.

In an embodiment, the growth models are dependent on at least one further/second parameter. In this content the at least one further/second parameter is preferably selected from the group: soil parameters of the location a hybrid breeding is planned, average intensity and duration of solar radiation at the location a hybrid breeding is planned, weather data, planned sowing density and/or planned harvest time. The different additional parameters may contribute to the growth model equations and describe some effects and/or interactions on the pollination phase and/or on the flowering phase. With the additional afore mentioned input parameters, also an accuracy of the computer-implemented growth model may increase. With the improved accuracy of the computer-implemented growth models, the prediction of the pollination phase and flowering phase improves and therefore the yield of the hybrid plant seeds increases. In this embodiment, the first computer-implemented growth model is configured to output time-resolved growth data of the male parent plant dependent on at least the first parameter and the at least one further/second parameter and the second computer-implemented growth model is configured to output time-resolved growth data of the female parent plant dependent on the at least first parameter and the at least one further/second parameter. In this embodiment the at least first parameter and the at least further/second parameter are inputted or provided into the first growth model to determine pollination phase data of the male parent plant and the at least first parameter and the at least one further/second parameter are inputted or provided into the second growth model to determine flowering phase data of the female parent plant. Thus, pollination phase data of the male parent plant and flowering phase data of the female parent plant based on the at least first parameter and the at least one further/second parameter can be provided.

In an embodiment, the growth models are statistical models and/or provided by use of a machine-learning algorithm. The statistical models may comprise Design of Experiments, linear and non-linear dependencies. The statistical models may be based on experimental data collected from running seasons in the past. By applying machine-learning algorithms (e.g. neuronal networks), the accuracy of the computer-implemented growth model may be advantageously increased. By usage of experimental data collected from running seasons in the past, the reliability of the computer-implemented growth model is advantageously high.

In an embodiment, the method further comprises the steps of providing treatment instructions. The treatment instructions guide the farmer in order synchronize the flowering phase and the pollination phase for an increase of yield of hybrid plant seeds. In this content, the treatment instructions are preferably selected form the group: sowing time, sowing density, use of plant growth regulators and/or herbicides, flower fertilization measures, seed treatment measures, use of sail fertilizers and/or use of fungicides/insecticides. The treatment instructions comprise nearly all parameters respectively aspects of planting and may therefore result in a high yield of the hybrid plant seed. The treatment instructions may also comprise measures to prolongate the flowering phase, which also increases the yield of the hybrid plants seeds.

In an embodiment, the method further comprises the step of obtaining weather data and/or soil data based on the location data at which hybrid breeding is planned. The weather data and/or soil data may be acquired from a data base or a user input. The weather data may comprise humidity data, temperatures, rainfall data, average amount of sunny days etc. The soil data may comprise pH values, density of the soil, nutrition values, nitrogen content, phosphorus content, average soil quality, altitude of the field etc. With these specific weather data and/or soil data, the accuracy of the computer-implemented growth model may increase and therefor the yield of hybrid plant seeds.

In an embodiment the parent plants are dioecious plants or monoecious plants, wherein the monoecious plants are preferably modified such that the monoecious plants dominantly express male or female fertility; and wherein the parent plants are preferably wheat plants, oilseed rape or rice plants modified such that they dominantly express male or female fertility.

The term dioecious means in this content, that the plants have only male reproduction organs or female reproduction organs. If the hybrid plant seeds are generated by ideal dioecious plants, the hybrid plants show advantageously a higher vigor and resilience against adverse environmental conditions. This is caused by the heterosis effect, which is mostly limited to the F1-generation. Hence, by usage of dioecious plants as paternal plants for generating of the hybrid plant seeds, the yield of the hybrid plant is advantageously increased compared to conventional plants and/or plant seeds from the next F2-generation. The term monoecious means in the present case that the plants have both male and female reproduction organs. Wheat, rice and oilseed rape are monoecious plants. Preferably the female parent plant of the monoecious plant is modified such that it only expresses female fertility, e.g. by sterilizing the male reproduction organs by use of heterologous cytoplasm. Hence, no self-pollination of the female parent plant can occur and the seeds generated at the female parent plant may be hybrid seeds.

In an embodiment, the flowering phase of the female parent plant and pollination phase of the male parent plant are different. Due to the highly genetically diversity of the parent plants, the pollination phase of the male parent plant and the flowering phase of the female parent plant are different. These different pollination phase and flowering phase may lead advantageously to a hybrid plant with a high heterosis effect and therefor to high vigor and/or resilience against adverse environmental conditions compared to conventional plants.

In an embodiment a method is provided, wherein the growth models may be or may also be based/dependent on data of treatments applied or intended/planned to be applied to the male and/or female parent plants. The term data of treatment applied to the male and/or female parent plants is in the present case to be understood broadly and comprises any data related to a treatment of the plants. In an example, the term data of treatment preferably comprises in the present case one or more of: sowing time, sowing density, use of plant growth regulators and/or herbicides, flower fertilization measures, seed treatment measures, use of soil fertilizers and/or use of fungicides/insecticides and/or time and amount of watering. The data of treatments applied to the male and/or female parent plants may also be provided from the farmer himself after a respective treatment. This may be advantageous to refine the growth models and therefor to increase the yield of hybrid seed.

In an embodiment a method is provided, wherein the growth models may be or may also be based/dependent on sensor based input data. The term sensor based input data may comprises any input data that are directly or indirectly obtained from sensors and that relate to conditions of the parent plants and/or an environment of the parent plants. In this context sensor based input data may preferably be obtained from a weed sensor, a disease sensor, a biomass sensor, a nitrate sensor, satellite imaging (e.g. for biomass assessment) and/or drone imaging (e.g. for biomass assessment). The sensor based input data may also be indirectly provided from a data base (e.g. central cloud based biomass data base). The sensor based input data may also be provided continuously and/or automated. This may be advantageous to refine the growth models and therefor to increase the yield of hybrid seed.

A further aspect of the present disclosure refers to a use of a computer-implemented growth model for a parent plant, wherein the computer-implemented growth model is configured to output time-resolved growth data of the parent plant dependent on at least a parameter in a method for determining plant data and/or for issuing treatment instructions in hybrid breeding. The computer-implemented growth model serves as basis for determination of the flowering phase and pollination phase of the parent plants as well as basis for the derivation of instructions for the farmer to increase the yield of hybrid plant seeds.

A further aspect of the present disclosure refers to a use of geographical location data of a field at which a hybrid breeding is planned in a method for determining plant data and/or for issuing treatment instructions in hybrid breeding. The geographical location data is an important information, which serves as input information for the computer-implemented growth model and is further associated with other parameters of the computer-implemented growth models in the method for determining plant data and/or for issuing treatment instructions in hybrid breeding. The use of geographical location data results in an advantageously reliability of the computer-implemented growth model due to an increased comparability with experimental training data from which the computer-implemented growth model is derived.

A further aspect of the present disclosure refers to a system for determining plant data and/or for issuing treatment instructions in hybrid breeding, comprising: at least one processing unit configured to process a first computer-implemented growth model for a male parent plant, wherein the first computer-implemented growth model is configured to output time-resolved growth data of the male parent plant dependent on at least a first parameter; at least one processing unit configured to process a second computer-implemented growth model for a female parent plant, wherein the second computer-implemented growth model is configured to output time-resolved growth data of the female parent plant dependent on the at least first parameter; at least one processing unit configured to input or provide the at least one parameter into the first growth model to determine pollination phase data of the male parent plant; at least one processing unit configured to input or provide the at least one parameter into the second growth model to determine flowering phase data of the female parent plant; at least one processing unit configured to provide pollination phase data of the male parent plant and flowering phase data of the female parent plant based on the at least first parameter. The processing unit might be in a smart phone, a tablet, a desktop computer, or a virtual machine stored in a cloud application. The system may be used from a farmer and may guide him to increase the yield of hybrid plant seed. The system may advantageously increase efficiency of hybrid plant seed generating.

A further aspect of the present disclosure refers to a computer program element which when executed by a processor in a system for determining plant data and/or for issuing treatment instructions in hybrid breeding is configured to carry out a method for determining plant data and/or for issuing treatment instructions in hybrid breeding. The computer program element might therefore be stored on a computing unit, which might also be part of an embodiment. This computing unit may be configured to perform or induce performing of the steps of the method described above. Moreover, it may be configured to operate the components of the above described system. The computing unit can be configured to operate automatically and/or to execute the orders of a user. A computer program may be loaded into a working memory of a data processor. The data processor may thus be equipped to carry out the method according to one of the preceding embodiments. This exemplary embodiment of the present disclosure covers both, a computer program that right from the beginning uses the present disclosure and computer program that by means of an update turns an existing program into a program that uses the present disclosure. Moreover, the computer program element might be able to provide all necessary steps to fulfill the procedure of an exemplary embodiment of the method as described above. According to a further exemplary embodiment of the present disclosure, a computer readable medium, such as a CD-ROM, USB stick or the like, is presented wherein the computer readable medium has a computer program element stored on it which computer program element is described by the preceding section. A computer program may be stored and/or distributed on a suitable medium, such as an optical storage medium or a solid state medium supplied together with or as part of other hardware, but may also be distributed in other forms, such as via the internet or other wired or wireless telecommunication systems. However, the computer program may also be presented over a network like the World Wide Web and can be downloaded into the working memory of a data processor from such a network. According to a further exemplary embodiment of the present disclosure, a medium for making a computer program element available for downloading is provided, which computer program element is arranged to perform a method according to one of the previously described embodiments of the present disclosure.

In the following, a non-exhaustive list of non-limiting examples is provided as embodiments A to O. Any one or more of the features of these examples may be combined with any one or more features of another example, embodiment, or aspect described herein.
A. Computer-implemented method for determining plant data and/or for issuing treatment instructions in hybrid breeding, comprising the following steps:
   providing a first computer-implemented growth model for a male parent plant, wherein the first computer-implemented growth model is configured to output time-resolved growth data of the male parent plant dependent on at least a first parameter (S10);
   providing a second computer-implemented growth model for a female parent plant, wherein the second computer-implemented growth model is configured to output time-resolved growth data of the female parent plant dependent on the at least first parameter (S10);
   inputting the at least one parameter into the first growth model to determine pollination phase data of the male parent plant (S20);
   inputting the at least one parameter into the second growth model to determine flowering phase data of the female parent plant (S20);
   providing pollination phase data of the male parent plant and flowering phase data of the female parent plant based on the at least first parameter (S30).
B. Method according to embodiment A, wherein the method further comprises the steps of providing plant data for the male parent plant and providing plant data for the female parent plant such that the flowering phase of the female plant corresponds to the pollination phase of the male plant (S40).
C. Method according to embodiment A or embodiment B, wherein the first parameter are geographic location data at which hybrid breeding is planned.
D. Method according to any one of the preceding embodiments, wherein the growth models are dependent on at least one further parameter.
E. Method according to embodiment D, wherein the at least one further parameter is selected from the group: soil parameters of the location a hybrid breeding is planned, average intensity and duration of solar radiation at the location a hybrid breeding is planned, weather data, planned sowing density and/or planned harvest time.
F. Method according to any one of the preceding embodiments, wherein the growth models are statistical models and/or provided by use of a machine-learning algorithm.
G. Method according to any one of the preceding embodiments, wherein the method further comprises the steps of providing treatment instructions (S50).
H. Method according to embodiment G, wherein treatment instructions are selected form the group: sowing time, sowing density, use of plant growth regulators and/or herbicides, flower fertilization measures, seed treatment measures, use of sail fertilizers and/or use of fungicides/insecticides.
I. Method according to any one of the embodiments B to H, wherein the method further comprises the step of obtaining weather data and/or soil data based on the location data at which hybrid breeding is planned.
J. Method according to any one of the preceding embodiments, wherein the parent plants are dioecious plants or monoecious plants, wherein at least one of the monoecious parent plant is preferably modified such that at least one of the monoecious parent plant dominantly express only male or female fertility; and wherein the parent plants are preferably wheat plants having female parent plants where the male reproduction organs are sterilized, preferably by use of a heterologous cytoplasm and/or a mutated gene and male parent plants having both female and male reproduction organs which both provide fertility.
K. Method according to any one of the preceding embodiments, wherein the flowering phase of the female parent plant and pollination phase of the male parent plant are different.
L. Use of a computer-implemented growth model for a parent plant, wherein the computer-implemented growth model is configured to output time-resolved growth data of the parent plant dependent on at least a parameter in a method according to any one of embodiments A to K.
M. Use of geographical location data of a field at which a hybrid breeding is planned in a method according to any one of embodiments A to K.
N. A system for determining plant data and/or for issuing treatment instructions in hybrid breeding, comprising:
   at least one processing unit configured to process a first computer-implemented growth model for a male parent plant, wherein the first computer-implemented growth model is configured to output time-resolved growth data of the male parent plant dependent on at least a first parameter;
   at least one processing unit configured to process a second computer-implemented growth model for a female parent plant, wherein the second computer-implemented growth model is configured to output time-resolved growth data of the female parent plant dependent on the at least first parameter;
   at least one processing unit configured to input the at least one parameter into the first growth model to determine pollination phase data of the male parent plant;
   at least one processing unit configured to input the at least one parameter into the second growth model to determine flowering phase data of the female parent plant;
   at least one processing unit configured to provide pollination phase data of the male parent plant and flowering phase data of the female parent plant based on the at least first parameter.
O. A computer program element which when executed by a processor in a system according to embodiment N is configured to carry out a method according to any one of embodiments A to K.

### BRIEF DESCRIPTION OF THE DRAWINGS

In the following, the present disclosure is described exemplarily with reference to the enclosed figure, in which

FIG. 1 is a schematic view of a method according to the preferred embodiment of the present disclosure; and

FIG. 2 illustrates an exemplary distributed system according to the present disclosure for treating hybrid breeding on an agricultural field.

### DETAILED DESCRIPTION OF EMBODIMENTS

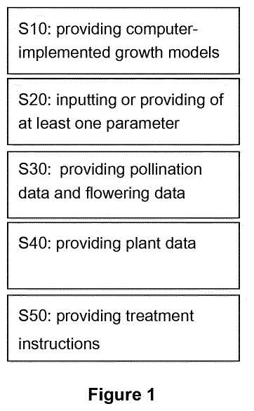

FIG. 1 is a schematic view of a method according to the preferred embodiment of the present disclosure. In the following, an exemplary order of the steps according to the present disclosure is explained. However, the provided order is not mandatory, i.e. all or several steps may be performed in a different order or simultaneously.

The method described below can be summarized as follows. In a preparatory step two computer-implemented growth models for a male parent plant (e.g. wheat) and a female parent plant (e.g. wheat) are provided. In a next step, a user (e.g. farmer) inputs at least one parameter (e.g. geographical location data) for example in his mobile, wherein the method runs. Afterwards in a next step the user is provided with pollination data of the male parent plant, flowering data of female parent plant, plant data and treatment data for both female parent plant and male parent plant on his mobile phone.

In a step S10, a first computer-implemented growth model for a male parent plant and a second computer-implemented growth model for a female parent plant are provided. Both computer-implemented growth models describe the relations between at least one input parameter (e.g. geographical location data at which the hybrid breeding is planned, soil parameters, average intensity and duration of solar radiation, weather data, planned sowing density and/or planned harvesting time) and the time-resolved growth data of the male parent plant and the female parent plant. The correlation may for example be derived by statistical methods (e.g. design of experiments) and machine learning algorithms (e.g. neuronal networks) from experimental data of seasons of the past. The computer-implemented growth models may be developed continuously by usage of the computer-implemented growth model in current running season. The computer-implemented growth model equations may comprise additional parameters like weather data (e.g. humidity data, temperatures, rainfall data, average amount of sunny days etc.) and soil data (e.g. may pH values, density of the soil, nutrition values, nitrogen content, phosphorus content, average soil quality etc.). The computer-implemented growth models may comprise categories for each parameter, wherein some categories have more or less effect on the time-resolved growth data. The computer-implemented growth models are each different for each specific female parent plant and male parent plant. The computer-implemented growth models may be stored and processed in a system for determining plant data and/or for issuing treatment instructions in hybrid breeding. The system may run on a tablet, mobile phone, desktop computer or a cloud application etc.

In a particularly preferred example, a male parent plant may be wheat with both male reproduction organs and female reproduction organs, and a female parent plant may be a wheat with both male reproduction organs and female reproduction organs. In this particularly preferred example the male reproduction organs and female reproduction organs of the male parent plant may express both fertility. The male reproduction organs of the female plant may be sterilized by use of a heterologous cytoplasm or a mutated gene. The female reproduction organs of the female may express fertility. In this particularly preferred example the first computer-implemented growth model relates to the male parent plant, in this case the wheat with both male reproduction organs and female reproduction organs, wherein both male reproduction organs and female reproduction organs provide fertility. In this particularly preferred example the second computer-implemented growth model relates to the female parent plant, in this case the wheat with both male reproduction organs and female reproduction organs, wherein the male reproduction organs are sterilized and the female reproduction organs provide fertility. The female parent plant may be planted in a row and the male parent plant may be planted in a further row, wherein the both rows are parallel to each other. In this particularly preferred example self-pollination may occur at the male parent plants and therefor leads to none hybrid seeds at the male parent plants. Self-pollination does not occur at the female parent plants. The female parent plants are pollinated from male parent plants, which leads to hybrid seeds. Hybrid seed is the seed harvested on the female parent plants. Hence, only a certain share of a yield of the female parent plants and male parent plants leads to hybrid seeds, e.g. in this example 50% hybrid seeds by 50% female parent plants and 50% male parent plants. In this respect, it is the aim that at least a certain yield is hybrid seed, e.g. 50%.

In a step S20 a user (e.g. a farmer or breeder) inputs at least one parameter (e.g. geographical location data at which the hybrid breeding is planned, soil parameters, average intensity and duration of solar radiation, weather data, planned sowing density and/or planned harvesting time) for a male parent plant and a female parent plant via a graphical user interface into the system. A processing unit (e.g. a CPU of tablet, mobile phone, desktop computer) processes then the at least one input parameter by using the computer-implemented growth model equations of the female parent plant and of the male parent plant and determines the flowering phase of the female parent plant and the pollination phase of the male parent plant. The determination of the time-resolved pollination phase and flowering phase may take into account different categories of the input parameters and/or computer-implemented growth model parameters that have either a higher or a lower effect on the time-resolved pollination phase and flowering phase. The effect of the input data may be determined by comparing the input data with optimal growth data conditions (i.e. different category of input data).

In a step S30 the time-resolved pollination phase data of the male parent plant and the time-resolved flowering phase data of the female parent plant are provided to a user (e.g. farmer or breeder). The providing may be carried out via figures or tables on a display of a tablet, mobile phone desktop computer or web browser.

In a step S40, plant data for the male parent plant and the female parent plant is provided to a user (e.g. farmer or breeder). The plant data is derived from a data base, wherein plant data (general sowing time, general flowering time, general pollination time, content of the plants, light requirement, temperature requirement, humidity requirement, nutrition requirement, plant species etc.) for each specific plant is stored. The plant data may be advantageous for a user to get a first information of the plants (e.g. approximate information of sowing time of the female parent plant and the male parent plant, in order to synchronize flowering and pollination phase, suitability combination plant/field). The plant data may serve as an overview in a planning phase for a user (e.g. farmer, breeder). The providing may be carried out via figures or tables on a display of a tablet, mobile phone desktop computer or web browser.

In step S50, treatment instruction data for the male parent plant and the female parent plant is provided to a user (e.g. farmer or breeder). The instruction data is derived from computer-implemented growth model. The instruction data guides the user to use appropriate measures to synchronize the flowering phase and the pollination phase and/or to prolongate the flowering phase. The instructions may comprise measures like applying plant growth regulators or herbicides, performing artificial flower fertilization steps (e.g. inducing wind flux such as by using drones), performing seed-treatment measures, using soil fertilizers, applying of fungicides/insecticides and/or determining harvesting times. The providing of the treatment instruction may be carried out via texts, figures, or tables on a display of a tablet, mobile phone desktop computer or web browser.

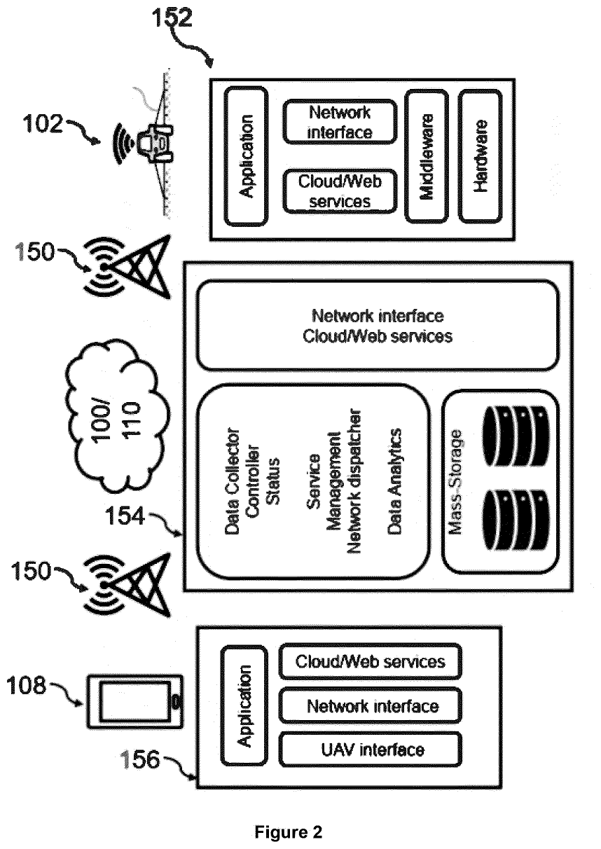

The system illustrated in FIG. 2 shows an exemplary distributed system including an agricultural vehicle 102 (e.g. a tractor for fertilizer spreading), which has been loaded/filled with an agricultural product, e.g. a fertilizer, one or more ground station(s) 110, one or more user device(s) 108, and a cloud environment 100. The agricultural vehicle 102 may be a manned or unmanned vehicle which can be controlled autonomously by onboard computers, remotely by a person or partially remotely e.g. by way of initial operation data. The agricultural vehicle 102 may transmit data signals collected from various onboard sensors and actors mounted to the agricultural vehicle 102. Such data may include current movement data such as current speed, battery or fuel level, position, weather or wind speed, field data including treatment operation data such as treatment type, treatment location or treatment mode, monitoring operation data such as field condition data or location data, and/or operation data, such as initial operation data, updated operation data or current operation data. The agricultural vehicle 102 may directly or indirectly send data signals, such as field data or operation data, to the cloud environment 100, the ground station(s) 110 or other agricultural vehicles (not shown). The agricultural vehicle 102 may directly or indirectly receive data signals, such as field data or operation data, from cloud environment 100, the ground station(s) 110 or other agricultural vehicles.

The cloud environment 100 may facilitate data exchange with and between the agricultural vehicle(s) 102, the ground control station(s) 110, and/or user device(s) 108. The cloud environment 100 may be a server-based distributed computing environment for storing and computing data on multiple cloud servers accessible over the Internet. The cloud environment 100 may be a distributed ledger network that facilitates a distributed immutable database for transactions performed by the agricultural vehicle 102, one or more ground station(s) 110 or one or more user device(s) 108. Ledger network refers to any data communication network comprising at least two network nodes. The network nodes may be configured to a) request the inclusion of data by way of a data block and/or b) verify the requested inclusion of data to the chain and/or c) receiving chain data. In such a distributed architecture, the agricultural vehicle(s) 102, one or more ground station(s) 110, one or more user device(s) 108 can act as nodes storing transaction data in data blocks and participating in a consensus protocol to verify transactions. If the at least two network nodes are in a chain the ledger network may be referred to as a blockchain network. The ledger network 100 may be composed of a blockchain or cryptographically linked list of data blocks created by the nodes. Each data block may contain one or more transactions relating to field data or operation data. Blockchain refers to a continuously extendable set of data provided in a plurality of interconnected data blocks, wherein each data block may comprise a plurality of transaction data. The transaction data may be signed by the owner of the transaction and the interconnection may be provided by chaining using cryptographic means. Chaining is any mechanism to interconnect two data blocks with each other. For example, at least two blocks may be directly interconnected with each other in the blockchain. A hash-function encryption mechanism may be used to chain data blocks in a blockchain and/or to attach a new data block in an existing blockchain. A block may be identified by its cryptographic hash referencing the hash of the preceding block.

The agricultural vehicle 102 and the ground control station(s) 103 may share data signals with the user device(s) 108 via the cloud environment 100. Communication channels between the nodes and communication channels, between the nodes and the cloud environment 100 may be established through a wireless communication protocol. A cellular network may be established for the agricultural vehicle 102 to ground station 110, other agricultural vehicles to cloud environment 100 or ground station 110 to cloud environment 100 communication. Such cellular network may be based any known network technology such as SM, GPRS, EDGE, UMTS /HSPA, LTE technologies using standards like 2G, 3G, 4G or 5G. In a local area of an agricultural field, a wireless local area network (WLAN), e.g. Wireless Fidelity (Wi-Fi), may be established for communication. The cellular network for may be a Flying Ad Hoc Network (FANET).

The present disclosure has been described in conjunction with a preferred embodiment as examples as well. However, other variations can be understood and effected by those persons skilled in the art and practicing the claimed invention, from the studies of the drawings, this disclosure and the claims. Notably, in particular the steps S10 to S50 can be performed in any order, i.e. the present invention is not limited to a specific order of these steps. Moreover, it is also not required that the different steps are performed at a certain place or at one place, i.e. each of the steps may be performed at a different place using different equipment/data processing units. In the claims as well as in the description the word “comprising” does not exclude other elements or steps and the indefinite article “a” or “an” does not exclude a plurality. A single element or other unit may fulfill the functions of several entities or items recited in the claims. The mere fact that certain measures are recited in the mutual different dependent claims does not indicate that a combination of these measures cannot be used in an advantageous implementation.

1. A method for determining plant data and/or for issuing treatment instructions in hybrid breeding, the method comprising:
providing a first computer-implemented growth model for a male parent plant, wherein the first computer-implemented growth model is configured to output time-resolved growth data of the male parent plant dependent on at least a first parameter (S10);
providing a second computer-implemented growth model for a female parent plant, wherein the second computer-implemented growth model is configured to output time-resolved growth data of the female parent plant dependent on the at least first parameter (S10);
inputting or providing at least one parameter into the first growth model to determine pollination phase data of the male parent plant (S20);
inputting or providing the at least one parameter into the second growth model to determine flowering phase data of the female parent plant (S20); and
providing pollination phase data of the male parent plant and flowering phase data of the female parent plant based on the at least first parameter (S30).

2. The method according to claim 1, wherein the method further comprises the steps of providing plant data for the male parent plant and providing plant data for the female parent plant such that the flowering phase of the female plant corresponds to the pollination phase of the male plant (S40).

3. The method according to claim 1, wherein the first parameter are geographic location data at which hybrid breeding is planned.

4. The method according to claim 1, wherein the growth models are dependent on at least one further/second parameter and wherein
the first computer-implemented growth model is configured to output time-resolved growth data of the male parent plant dependent on at least the first parameter (S10) and the at least one further/second parameter;
the second computer-implemented growth model is configured to output time-resolved growth data of the female parent plant dependent on the at least first parameter (S10) and the at least one further/second parameter;
inputting or providing the at least first parameter and the at least further/second parameter into the first growth model to determine pollination phase data of the male parent plant (S20);
inputting or providing the at least first parameter and the at least one further/second parameter into the second growth model to determine flowering phase data of the female parent plant (S20); and
providing pollination phase data of the male parent plant and flowering phase data of the female parent plant based on the at least first parameter (S30) and the at least one further/second parameter.

5. The method according to claim 4, wherein the at least one further/second parameter is selected from the group: soil parameters of the location at which hybrid breeding is planned, average intensity and duration of solar radiation at the location at which hybrid breeding is planned, weather data, planned sowing density and/or planned harvest time.

6. The method according to claim 1, wherein the growth models are statistical models and/or provided by use of a machine-learning algorithm.

7. The method according to claim 1, wherein the method further comprises providing treatment instructions (S50).

8. The method according to claim 7, wherein treatment instructions are selected form the group: sowing time, sowing density, use of plant growth regulators and/or herbicides, flower fertilization measures, seed treatment measures, use of soil fertilizers and/or use of fungicides/insecticides.

9. The method according to claim 2, wherein the method further comprises the step of obtaining weather data and/or soil data based on the location data at which hybrid breeding is planned.

10. The method according to claim 1, wherein the parent plants are dioecious plants or monoecious plants, wherein at least one of the monoecious parent plant is modified such that at least one of the monoecious parent plant dominantly express only male or female fertility; and wherein the parent plants are wheat plants having female parent plants where the male reproduction organs are sterilized by use of a heterologous cytoplasm and/or a mutated gene and male parent plants having both female and male reproduction organs which both provide fertility.

11. The method according to claim 1, wherein the flowering phase of the female parent plant and pollination phase of the male parent plant are different.

12. Use of a computer-implemented growth model for a parent plant, wherein the computer-implemented growth model is configured to output time-resolved growth data of the parent plant dependent on at least a parameter in a method according to claim 1.

13. Use of geographical location data of a field at which a hybrid breeding is planned in a method according to claim 1.

14. A system for determining plant data and/or for issuing treatment instructions in hybrid breeding, the system comprising:
at least one processing unit configured to process a first computer-implemented growth model for a male parent plant, wherein the first computer-implemented growth model is configured to output time-resolved growth data of the male parent plant dependent on at least a first parameter;
at least one processing unit configured to process a second computer-implemented growth model for a female parent plant, wherein the second computer-implemented growth model is configured to output time-resolved growth data of the female parent plant dependent on the at least first parameter;
at least one processing unit configured to input or provide the at least one parameter into the first growth model to determine pollination phase data of the male parent plant;
at least one processing unit configured to input or provide the at least one parameter into the second growth model to determine flowering phase data of the female parent plant; and
at least one processing unit configured to provide pollination phase data of the male parent plant and flowering phase data of the female parent plant based on the at least first parameter.

15. A non-transitory computer readable medium having instructions encoded thereon that, when executed by a processing unit, cause the processing unit to carry out the method according to claim 1.

16. A method for the treatment of hybrid breeding, the method comprising:
providing treatment instructions according to claim 7;
providing control data for an agricultural equipment based on the provided treatment instructions; and
controlling the agricultural equipment using the provided control data.

17. Agricultural equipment controlled based on treatment instructions provided according to claim 7.

18. (canceled)
